(19) United States
(12) Patent Application Publication
(10) Pub. No.: US 2023/0116647 A1
(43) Pub. Date: Apr. 13, 2023

(54) METHODS OF TREATING COVID-19 WITH RIFAXIMIN

(71) Applicant: Bausch Health Ireland Limited, Dublin (IE)

(72) Inventors: Maxime Barakat, Longueuil (CA); Howard Franklin, Richboro, PA (US); Zeev Heimanson, Calabasas, CA (US); Thejasvi Ramakrishna, Flemington, NJ (US); Jason Lamar Vittitow, Flanders, NJ (US)

(21) Appl. No.: 17/913,843

(22) PCT Filed: Mar. 24, 2021
(86) PCT No.: PCT/EP2021/057641

§ 371 Date: Sep. 23, 2022

Related U.S. Application Data
(60) Provisional application No. 63/034,197, filed on Jun. 3, 2020.
(60) Provisional application No. 62/994,009, filed on Mar. 24, 2020.

(51) Int. Cl. A61K 31/437; A61K 45/06; A61K 31/4706

(52) U.S. Cl. CPC A61K 31/437; A61K 45/06; A61K 31/4706

(57) ABSTRACT

Provided herein are methods for treating COVID-19 or associated GI symptoms with rifaximin compositions.

### RELATED APPLICATIONS

This application claims priority to U.S. Provisional Application No. 62/994,009, filed, Mar. 24, 2020 and U.S. Provisional Application No. 63/034,197, filed Jun. 3, 2020, the entire contents of each of which are incorporated herein by reference.

### FIELD OF THE INVENTION

The present disclosure relates generally to therapies for treating coronaviruses such as COVID-19 and symptoms associated therewith.

### BACKGROUND OF THE INVENTION

COVID-19 is a Betacoronavirus similar to the Severe Acute Respiratory Syndrome Human coronavirus (SARS HCoV) and the Middle-East Respiratory Syndrome Human coronavirus (MERS HCoV). Six different strains of Human coronaviruses (HCoVs) have been reported, in addition to the newly emerged COVID-19. SARS and MERS HCoV are the most aggressive strains of coronaviruses, responsible for approximately 800 deaths each. SARS HCoV has a 10% mortality rate, while MERS HCoV has a 36% mortality rate, according to the WHO.

HCoVs generally are positive-sense and very long (30,000 bp) single-stranded RNA viruses. RNA dependent RNA polymerase (RdRp) is a crucial enzyme in the life cycle of RNA viruses. The active site of RdRp is highly conserved and is targeted in different RNA viruses, including Hepatitis C Virus (HCV), Zika Virus (ZIKV), and coronaviruses (CoVs).

Antiviral drugs approved against other viruses by the Food and Drugs Administration (FDA), such as Sofosbuvir, Ribavirin have been used against RdRp of Hepatitis C Virus (HCV). These drugs are nucleotides derivative competing with physiological nucleotide for RdRp active site. Additionally, a large number of attempts to develop anti-RdRp compounds are under clinical testing against different viruses. However, there are no approved therapies for COVID-19.

Accordingly, there is a desperate need in the field for COVID-19 therapies as well as for other coronaviruses.

### SUMMARY OF THE INVENTION

Recent published data indicate that close to 50% of patients infected with COVID-19 virus presented to the hospital with digestive symptoms as their chief complaint (Pan L. at al., AJG, 2020). As described herein, rifaximin (with or without an additional therapeutic agent) may be used for the treatment of COVID-19 or GI symptoms associated with COVID-19 in patients in need thereof.

In an embodiment, the invention described herein includes a method of treating a coronavirus or symptoms associates therewith in a patient in need thereof, where the method may include administering an amount of rifaximin to the patient. In some embodiments, the invention described herein includes a method of treating a gastrointestinal (GI) coronavirus symptom in a patient in need thereof, where the method may include administering an amount of rifaximin to the patient. In some embodiments, the invention described herein includes a method of treating a GI symptom associated with a coronavirus in a patient in need thereof, where the method may include administering an amount of rifaximin to the patient.

In some embodiments of the methods described herein, the coronavirus is COVID-19.

In some embodiments of the methods described herein, the amount of rifaximin administered is a therapeutically effective amount of rifaximin.

In some embodiments of the methods described herein, the rifaximin comprises rifaximin polymorph form α, rifaximin polymorph form β, rifaximin polymorph form γ, rifaximin polymorph form δ, rifaximin polymorph form ϵ, amorphous rifaximin, or a combination thereof.

In some embodiments of the methods described herein, the amount of rifaximin is about 1 mg to about 2500 mg, or about 50 mg to about 1000 mg, or about 150 mg to about 600 mg, or about 200 mg to about 550 mg, or about 200 mg, or about 550 mg. In some embodiments, the rifaximin is administered as a 200 mg rifaximin dosage form. In some embodiments, the rifaximin is administered as a 550 mg rifaximin dosage form.

In some embodiments of the methods described herein, the rifaximin is administered QD, BID, TID, or QID. In some embodiments, the rifaximin is administered TID.

In some embodiments, the methods described herein include the administration of at least one additional therapeutic agent. In some embodiments, the at least one additional therapeutic agent is administered concomitantly or sequentially with the rifaximin. In some embodiments, the at least one additional therapeutic agent includes an antiviral. In some embodiments, the antiviral may include one or more of azithromycin, hydroxychloroquine, and chloroquine. In some embodiments, the at least one additional therapeutic agent may be administered in an amount of about 100 mg to about 1000 mg, or about 200 mg to about 900 mg, or about 300 mg to about 800 mg, or about 400 mg to about 700 mg, or about 250 mg, or about 500 mg, or about 600 mg. In some embodiments, the at least one additional therapeutic agent may be administered QD, BID, TID, or QID. In some embodiments, the at least one additional therapeutic agent may be administered BID.

In some embodiments, the methods described herein may further include the administration of supplemental oxygen, noninvasive ventilation, invasive ventilation, additional antiviral agents, other antibiotic agents, vasopressor support, renal-replacement therapy, extracorporeal membrane oxygenation, or a combination thereof.

In some embodiments of the methods described herein, the rifaximin may be administered until clearance of viral infection based on a negative test for the coronavirus.

In some embodiments of the methods described herein, the rifaximin may be administered until the patient no longer exhibits coronavirus symptoms.

In some embodiments of the methods described herein, the rifaximin may be administered fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s).

In some embodiments of the methods described herein, the rifaximin may be administered after the patient no longer exhibits coronavirus symptoms for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional day(s).

In some embodiments, the methods described herein may include one or more of: (i) monitoring the patient's heart rhythm; and (ii) detecting irregularities in the patient's heart rhythm.

In some embodiments of the methods described herein, a GI symptom associated with coronavirus may be one or more of nausea, anorexia, diarrhea, vomiting, and abdominal pain. In some embodiments, the GI symptom associated with coronavirus may include diarrhea.

In some embodiments, the methods described herein include treating coronavirus and/or gastrointestinal (GI) coronavirus symptoms using rifaximin in patient who is already being treated for coronavirus and/or gastrointestinal (GI) coronavirus symptoms with a therapy other than rifaximin.

In some embodiments, the methods described herein include treating COVID-19 and/or gastrointestinal (GI) COVID-19 symptoms and/or symptoms associated using rifaximin as described herein in patient who is already being treated for COVID-19 and/or gastrointestinal (GI) COVID-19 symptoms with a therapy other than rifaximin.

In an embodiment, the invention described herein includes a kit for treating coronavirus and/or GI coronavirus symptoms and/or GI symptoms associated with coronavirus (e.g., COVID-19) in a patient in need thereof, wherein the kit contains rifaximin. In some embodiments, the invention described herein includes a kit for treating coronavirus and/or GI coronavirus (e.g., COVID-19) symptoms and/or GI symptoms associated with coronavirus (e.g., COVID-19) in a patient in need thereof, wherein the kit contains a therapeutically effective amount of rifaximin.

### DETAILED DESCRIPTION OF THE INVENTION

Unless defined otherwise, all technical and scientific terms used herein have the same meaning as is commonly understood by a person having ordinary skill in the art to which this invention pertains. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition, or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

As used herein, the term “pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range.

Furthermore, the transitional terms “comprising”, “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments described herein that encompass the invention can, in alternate embodiments, be more specifically defined by any of the transitional terms “comprising,” “consisting essentially of,” and “consisting of.”

### Rifaximin

Rifaximin (INN; see The Merck Index, XIII Ed., 8304) is an antibiotic belonging to the rifamycin class of antibiotics, e.g., a pyrido-imidazo rifamycin. Rifaximin exerts its broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Rifaximin tablets have been approved in the US in the following capacities: 200 mg tablets (XIFAXAN® [rifaximin] Tablets) thrice daily (TID) in May 2004 for the treatment of traveler's diarrhea (TD) caused by noninvasive strains of Escherichia coli in patients 12 years of age or older, 550 mg tablets (XIFAXAN) twice daily (BID) in March 2010 as chronic, daily therapy for the reduction in risk of overt hepatic encephalopathy (HE) recurrence in patients 18 years of age or older and 550 mg tablets (XIFAXAN) in March 2015 for treatment of irritable bowel syndrome with diarrhea (IBS-D) in adults.

Rifaximin may exist in a number of different solvate, hydrate, polymorphic, and/or crystalline forms, including form α, form β, form γ, form δ, form ϵ, and amorphous forms of rifaximin. In some embodiments, rifaximin as set forth herein may be form α, form β, form γ, form δ, form ϵ, amorphous, or a combination thereof. Forms, formulations, and methods of using rifaximin are described, for example, in U.S. Pat. Nos. 7,045,620, 7,906,542, 7,915,275, 8,193,196, 8,309,569, 8,518,949, and 8,741,904, the entirety of which are incorporated herein by reference. In some embodiments, rifaximin may be administered with, for example, a solid formulation (e.g., by tablet), a liquid formulation, and/or a soluble solid dispersion.

Rifaximin exerts a broad antibacterial activity in the gastrointestinal tract against localized gastrointestinal bacteria that cause infectious diarrhea, including anaerobic strains. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Without wishing to be bound by any particular scientific theories, rifaximin acts by binding to the beta-subunit of the bacterial deoxyribonucleic acid-dependent ribonucleic acid (RNA) polymerase, resulting in inhibition of bacterial RNA synthesis. It is active against numerous gram (+) and (−) bacteria, both aerobic and anaerobic. In vitro data indicate rifaximin is active against species of Staphylococcus, Streptococcus, Enterococcus, and Enterobacteriaceae. Bacterial reduction or an increase in antimicrobial resistance in the colonic flora does not frequently occur and does not have a clinical importance.

In the embodiments described herein, rifaximin may be provided in methods of treating disease and compositions for use in such methods. In the embodiments described herein, rifaximin may be provided in a therapeutically effective amount. In some embodiments, rifaximin may be provided in an amount of about 1 mg to about 2500 mg, or about 50 mg to about 1000 mg, or about 150 mg to about 600 mg, or about 200 mg to about 550 mg.

In some embodiments, rifaximin may refer to one or more Xifaxan® tablets. In some embodiments, rifaximin may refer to the 200 mg dosage form of Xifaxan® tablets. In some embodiments, rifaximin may refer to one or more tablets of the 200 mg dosage form of Xifaxan® tablets. In some embodiments, rifaximin may refer to the 550 mg dosage form of Xifaxan® tablets. In some embodiments, rifaximin may refer to one or more tablets of the 550 mg dosage form of Xifaxan® tablets.

### Coronaviruses (e.g., COVID-19)

The invention includes methods of treating coronaviruses such as COVID-19 and/or associated symptoms with rifaximin as described herein.

Diverse digestive symptoms have been regularly reported in patients infected with COVID-19. In a recently published study, 204 patients with COVID-19 were analyzed. GI symptoms were a main complaint in 48.5% of patients presented to the hospital. These patients had a significantly longer time from onset to admission than patients without digestive symptoms (9.0 days vs. 7.3 days). GI symptoms varied and included anorexia (83.8%), diarrhea (29.3%), vomiting (0.8%), and abdominal pain (0.4%). Of interest, in 7 patients, digestive symptoms were present without respiratory symptoms. There was a direct correlation between disease severity and GI symptoms. Patients without GI symptoms had superior outcomes—more likely to be cured and discharged from inpatient care than patients with GI symptoms 60% vs. 34.3%). (Pan L. et al. AJG, 2020.).

In a separate study of 73 hospitalized patients, 53.42% tested positive for COVID-19 RNA in stool (Xiao F. et al., Gastroenterology, 2020).

Without being limited to any particular theory, there are several theories regarding the pathophysiology of GI symptoms in patients with COVID-19 infection: (1) COVID-19 indirectly or directly damages the digestive system through an inflammatory response; (2) COVID-19 may directly damage the intestinal mucosa; and (3) COVID-19 may affect composition of the intestinal flora.

Furthermore, changes in the composition and function of GI microbiota may affect the respiratory tract through the common mucosal immune system. In turn, respiratory tract flora perturbation may affect the GI tract through immune regulation. This effect is termed “gut-lung axis” which may elucidate as to why patients with COVID-19 pneumonia may often exhibit GI symptoms. (Pan L. et al. AJG, 2020).

Additionally, COVID-19 is known to induce excessive and aberrant non-effective host immune responses that are associated with severe lung pathology, leading to death. Similar to patients with SARS-CoV and MERS-CoV, some patients with COVID-19 develop acute respiratory distress syndrome (ARDS) with characteristic pulmonary ground glass changes on imaging. In most moribund patients, COVID-19 infection is also associated with a cytokine storm. In those who survive intensive care, these aberrant and excessive immune responses lead to long-term lung damage and fibrosis, causing functional disability and reduced quality of life (Zumla et.al., 2020). Predictors of fatality from a recent retrospective, multicenter study of 150 confirmed COVID-19 cases in Wuhan, China, included elevated ferritin (mean 1297.6 ng/ml in non-survivors vs 614.0 ng/ml in survivors; p<0.001) and IL-6 (p<0.0001), suggesting that mortality might be due to virally driven hyper-inflammation (Ruan Q et al., Intensive Care Med. 2020.)

IL-6 is a pleiotropic cytokine often associated with inflammation. IL-6 is expressed in gastric and small intestinal epithelial cells. Within the intestine, IL-6 has been shown to prevent epithelial apoptosis during prolonged inflammation. (Kuhn et al., PLoS One. 2014.) Expression of IL-6 in colonic mucosa correlates quantitatively with the presence of its m-RNA. Shirota K et al., Virchows Arch B Cell Pathol Incl Mol Pathol. 1990) A study in mice showed that an excessive level of IL-6 produced following viral infection enhances the expression of survival molecules which hinder critical host defenses that are responsible for removing virus-infected cells. (Hou W, et al., J Virol. 2014).

In COVID-19 infected patients levels of IL-6 were elevated in non-survivors compared with survivors throughout the clinical course, and increased with illness deterioration. (Zhou F. et al., The Lancet. 2020).

Laboratory results of a retrospective multicenter study of 68 death cases compared to 82 discharged cases showed that there were significant differences in interleukin-6 (IL-6) between the two groups.

The role of IL-6 in viral GI disease was established in several trials. 50 children with bacterial diarrhea and 70 with viral diarrhea were studied. The levels of serum IL-6, PBMCs IL-6 mRNA transcription and IL-6 protein expression were higher in the viral infection group than in the bacterial infection group (all p<0.001). Furthermore, IL-6 is involved in the pathogenesis of acute gastroenteritis in both rotavirus and norovirus. Finally, the IL-6 concentration was increased in SARS, a closely related virus to COVID-19, and was significantly elevated in severe SARS patients.

Without being limited to any one theory, COVID-19 may directly damage the intestinal mucosa or may do so via its effect on the intestinal flora composition. The eubiotic effect of rifaximin has been shown in several studies of patients with GI conditions. Additionally, without being limited to any one theory, COVID-19 induced dysbiosis may be corrected by rifaximin administration. Furthermore, rifaximin may have an effect on suppression of IL-6 in diseases states that are associated with IL-6 elevation. Data exists on the association between reduction in IL-6 levels and symptomatic improvements in patients diagnosed with these conditions.

Several animal models show the effect of rifaximin on IL-6 and other inflammatory factors. Rifaximin was administered to the proteoglycan (PG)-induced ankylosing spondylitis mice for 4 consecutive weeks. Rifaximin significantly reduced the severity of ankylosing spondylitis and resulted in down-regulation of inflammatory factors, such as TNF-α, IL-6, IL-17A, and IL-23. (Yang L., Front Cell Infect Microbiol. 2019).

In a mouse model of Hepatic Encephalopathy, rifaximin improved systemic inflammation (serum IL-1β, IL-6), intestinal barrier (FITC-dextran, large-/small-intestinal expression of IL-1β, IL-6, MCP-1, e-cadherin and zonulin) along with microbiota composition and function. Rifaximin also favorably impacted microbiota function (reduced endotoxin and decreased deconjugation and formation of potentially toxic secondary bile acids) (Kang D. et al., Clin Transl Gastroenterol. 2016).

In cirrhotic patients with Minimal Hepatic Encephalopathy serum levels of IL-6 and IL-18 are higher than in patients without MHE and correlate with cognitive impairment. Rifaximin normalized all alterations of the immune system in responders (59%) while in non-responders it normalizes only IL-6, CCL20, and differentiation of T lymphocytes to Th22. Mangas-Losada A, et al., J Transl Med. 2019).

In a NASH study, patients were randomized to rifaximin or placebo. After 6 months of rifaximin therapy, patients with NASH showed a significant reduction in homeostatic model assessment, alanine aminotransferase, aspartate aminotransferase, γ-glutamyl transferase, endotoxin, toll-like receptor-4, IL-6, tumor necrosis factor-α, CK-18, and NAFLD-liver fat score (all P<0.05) (Abdel-Razik A, et al., Eur J Gastroenterol Hepatol. 2018).

Circulating IL-6 levels are higher in IBS patients compared to controls (p=0.01). Of note, IL-6 level is significantly higher in diarrhea predominant IBS compared to control (p=0.03). (Bashashati M, et al., Cytokine. 2017). Furthermore, the inflammatory parameters and markers of bacterial translocation (IL-6 and LPS) were significantly higher in patients with diarrhea predominant IBS. (Linsalata M BMC Gastroenterol. 2018).

Rifaximin is presently indicated for treatment of IBS with diarrhea. The effect of rifaximin on potential inflammatory mediators was investigated in a rat model for visceral hyperalgesia. Rifaximin decreased the overall quantity of bacteria and altered the bacterial composition in the ileum with a higher relative abundance of Lactobacillus species. Rifaximin decreased mucosal inflammation, measured by decreased levels of interleukins (IL-17 and IL-6) and tumor necrosis factor α (TNF-α). Of note, active comparator, neomycin, did not decrease visceral pain and did not prevent the elevation in IL-17, IL-6, or TNF-α. (Xu D, et al., Gastroenterology. 2014).

Armed with the foregoing information, rifaximin may be used to treat COVID-19 or symptoms associated therewith in patients in need thereof.

### Methods of Treatment

One or more rifaximin compositions described herein may be used for the treatment of coronaviruses such as COVID-19 and/or symptoms thereof.

In an embodiment, the invention includes a method of treating coronaviruses such as COVID-19 in a patient in need thereof with a therapeutically effective amount of rifaximin.

In an embodiment, the invention includes a method of treating GI symptoms associated with coronaviruses such as COVID-19 in a patient in need thereof, comprising administering a therapeutically effective amount of rifaximin.

In an embodiment, the invention includes a method of treating a GI symptom associated with COVID-19 in a patient in need thereof, comprising administering a therapeutically effective amount of rifaximin. In some embodiments, the GI symptom is selected from the group consisting of nausea, anorexia, diarrhea, vomiting, and abdominal pain. In some embodiments, the GI symptom is diarrhea.

In an embodiment, the invention includes a method of treating diarrhea associated with COVID-19 in a patient in need thereof, comprising administering a therapeutically effective amount of rifaximin.

In some embodiments of the foregoing methods, the administration of a therapeutically effective amount of rifaximin may be in addition to another COVID-19 treatment regimen prescribed to the patient. In some embodiments, the patient is already undergoing treatment for COVID-19 with a therapy other than rifaximin prior to the administration of rifaximin.

In some embodiments of the foregoing methods, rifaximin may be administered to a patient in need thereof in an amount of about 1 mg to about 2500 mg, or about 50 mg to about 1000 mg, or about 150 mg to about 600 mg, or about 200 mg to about 550 mg, or about 200 mg, or about 550 mg.

In some embodiments of the foregoing methods, rifaximin may be administered QD, BID, TID, or QID.

In some embodiments, rifaximin may be administered in solid form (e.g., tablets) or in a liquid form (e.g., as a slurry mixed with a liquid vehicle such as water).

In some embodiments of the foregoing methods, rifaximin may be one or more tablets of a 200 mg rifaximin dosage form (e.g., Xifaxan® 200 mg tablets).

In some embodiments of the foregoing methods, rifaximin may be one or more tablets of a 550 mg rifaximin dosage form (e.g., Xifaxan® 550 mg tablets).

In some embodiments, the foregoing methods may include the further administration of a therapeutically effective amount of one or more additional therapeutic agents such as, for example, an antiviral and/or azithromycin. In some embodiments, the one or more additional therapeutic agents may be provided in an amount of about 100 mg to about 1000 mg, or about 200 mg to about 900 mg, or about 300 mg to about 800 mg, or about 400 mg to about 700 mg, or about 250 mg, or about 500 mg, or about 600 mg. In some embodiments, the one or more additional therapeutic agents may be provided QD, BID, TID, or QID. In some embodiments, the one or more additional therapeutic agents may be provided concomitantly or sequentially with the rifaximin.

In some embodiments, the additional therapeutic agent includes an antiviral, such as hydroxychloroquine and/or chloroquine, which may be provided in an amount of about 100 mg to about 1000 mg, or about 200 mg to about 900 mg, or about 300 mg to about 800 mg, or about 400 mg to about 700 mg, or about 500 mg or about 600 mg. In some embodiments, the hydroxychloroquine or chloroquine may be provided QD, BID, TID, or QID. In some embodiments, the hydroxychloroquine or chloroquine may be provided concomitantly or sequentially with the rifaximin. In an embodiment, the additional therapeutic agent includes hydroxychloroquine provided at dosage of about 600 mg. In an embodiment, the additional therapeutic agent includes chloroquine provided at a dosage of about 500 mg.

In some embodiments, the additional therapeutic agent includes azithromycin, which may be provided in an amount of about 100 mg to about 1000 mg, or about 200 mg to about 900 mg, or about 300 mg to about 800 mg, or about 400 mg to about 700 mg, or about 250 mg, about 500 mg. In some embodiments, the azithromycin may be provided QD, BID, TID, or QID. In some embodiments, the azithromycin may be provided at a dosage of 500 mg on the first day of therapy according to the methods of the invention and then at a dosage of 250 mg for each day thereafter. In some embodiments, the azithromycin may be provided concomitantly or sequentially with the rifaximin.

In some embodiments, the foregoing methods include administration of 550 mg rifaximin TID and one or more additional therapeutic agents selected from the group consisting of hydroxychloroquine, chloroquine, azithromycin, and a combination thereof.

In some embodiments, the foregoing methods include administration of 500 mg chloroquine BID and 550 mg rifaximin TID.

In some embodiments, the foregoing methods may include the further administration of supplemental oxygen, noninvasive ventilation, invasive ventilation, additional antiviral agents, other antibiotic agents, vasopressor support, renal-replacement therapy, extracorporeal membrane oxygenation, or a combination thereof.

In an embodiment, the invention includes a method of treating coronaviruses such as COVID-19 in a patient in need thereof, comprising administering 200 mg rifaximin or 550 mg rifaximin. In some embodiments, the 200 mg rifaximin is administered BID or TID. In some embodiments, the 550 mg rifaximin is administered BID or TID. In some embodiments, the method includes administering 500 mg chloroquine BID and 550 mg rifaximin TID.

In an embodiment, the invention includes a method of treating GI symptoms associated with coronaviruses such as COVID-19 in a patient in need thereof, comprising administering 200 mg rifaximin or 550 mg rifaximin. In some embodiments, the 200 mg rifaximin tablets are administered BID or TID. In some embodiments, the 550 mg rifaximin is administered BID or TID. In some embodiments, the method includes administering 500 mg chloroquine BID and 550 mg rifaximin TID.

In an embodiment, the invention includes a method of treating coronaviruses such as COVID-19 in a patient in need thereof, comprising administering (a) one or more 200 mg rifaximin tablets or one or more 550 mg rifaximin tablets, and (b) a therapeutically effective amount of chloroquine. In some embodiments, the 200 mg rifaximin tablets are administered BID or TID. In some embodiments, the 550 mg rifaximin tablets are administered BID or TID. In some embodiments, the chloroquine is provided in an amount of about 500 mg. In some embodiments, the chloroquine is administered BID or TID. In some embodiments, the method includes administering 500 mg chloroquine tablets BID and 550 mg rifaximin tablets TID.

In an embodiment, the invention includes a method of treating GI symptoms associated with coronaviruses such as COVID-19 in a patient in need thereof, comprising administering (a) one or more 200 mg rifaximin tablets or one or more 550 mg rifaximin tablets, and (b) a therapeutically effective amount of chloroquine. In some embodiments, the 200 mg rifaximin tablets are administered BID or TID. In some embodiments, the 550 mg rifaximin tablets are administered BID or TID. In some embodiments, the chloroquine is provided in an amount of about 500 mg. In some embodiments, the chloroquine is administered BID or TID. In some embodiments, the method includes administering 500 mg chloroquine tablets BID and 550 mg rifaximin tablets TID.

In an embodiment, the invention includes a method of treating coronaviruses such as COVID-19 in a patient in need thereof, comprising administering 500 mg chloroquine tablets BID and 550 mg rifaximin tablets TID.

In some embodiments of the foregoing methods of the invention, the administration of rifaximin with or without an additional therapeutic agent may continue until clearance of viral infection based on a negative test for coronaviruses such as COVID-19.

In some embodiments of the foregoing methods of the invention, the administration of rifaximin with or without an additional therapeutic agent may continue until the patient no longer exhibits coronavirus symptoms such as COVID-19 symptoms or, more specifically, GI symptoms.

In some embodiments of the foregoing methods of the invention, the administration of rifaximin with or without an additional therapeutic agent may continue for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s).

In some embodiments of the foregoing methods of the invention, the administration of rifaximin with or without an additional therapeutic agent may continue after the patient no longer exhibits coronavirus symptoms such as COVID-19 symptoms or, more specifically, GI symptoms for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional day(s).

In an embodiment, the invention includes the treatment of a GI symptom (e.g., diarrhea) associated with COVID-19 in a patient (who may or may not be taking an additional therapeutic agent or regimen for treating COVID-19), comprising administering a 550 mg of rifaximin TID fora period of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days (e.g., 14 days).

In some embodiments of the foregoing methods of the invention, the administration of rifaximin with or without an additional therapeutic agent may be performed on an inpatient or outpatient basis.

In some embodiments, the methods described herein further include monitoring the patient's heart rhythm and/or detecting irregularities in the patient's heart rhythm. In some embodiments, the methods described herein further includes performing an electrocardiogram to monitor the patient's heart rhythm and/or detect irregularities in the patient's heart rhythm.

### Kits

The invention described herein also provides kits for delivering the methods described herein. The kits include a dose of rifaximin (and any additional therapeutic agents) in suitable packaging, and written material that can include instructions for use in treating coronaviruses such as COVID-19 and/or symptoms associated therewith, discussion of clinical studies and listing of side effects. Such kits may also include information, such as scientific literature references, package insert materials, clinical trial results, and/or summaries of these and the like, which indicate or establish the activities and/or advantages of the compositions, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like.

While certain embodiments of the present invention have been described and/or exemplified above, various other embodiments will be apparent to those skilled in the art from the foregoing disclosure. The invention is, therefore, not limited to the particular embodiments described and/or exemplified but is capable of considerable variation and modification without departure from the scope and spirit of the appended claims.

### EXAMPLES

The following examples describe the invention in further detail. These examples are provided for illustrative purposes only and should in no way be considered as limiting the invention.

### Example 1: Protocol for Treating COVID-19 Patients with Xifaxan® tablets (Rifaximin 550 mg Tablets)

The benefits of a rifaximin therapy for treating COVID-19 in patients may be demonstrated with a clinical trial according to the following protocol.

Inclusion criteria: Hospitalized patients ≥18 years of age diagnosed with COVID-19 infection.

Treatment arms: (1) chloroquine (500 mg tablets BID); and (2) chloroquine (500 mg tablets BID) and rifaximin (550 mg tablets TID). Both arms to receive standard of care as needed that may include supplemental oxygen, noninvasive and invasive ventilation, antibiotic agents, vasopressor support, renal-replacement therapy, and extracorporeal membrane. oxygenation

Primary End Point: Time to clinical improvement defined as the time from randomization to an improvement of two points (from the status at randomization) on a seven-category ordinal scale or live discharge from the hospital, whichever came first. The seven-category ordinal scale consists of the following categories: 1, not hospitalized with resumption of normal activities; 2, not hospitalized, but unable to resume normal activities; 3, hospitalized, not requiring supplemental oxygen; 4, hospitalized, requiring supplemental oxygen; 5, hospitalized, requiring nasal high-flow oxygen therapy, noninvasive mechanical ventilation, or both; 6, hospitalized, requiring ECMO, invasive mechanical ventilation, or both; and 7, death.

Secondary End Points: (1) mortality, (2) clearance of viral infection based on a negative test for COVID-19 (serial oropharyngeal swab samples on day 1 (before drug was administered) and on days 5, 10, 14, 21, and 28 until discharge or death had occurred), (3) time to ICU discharge, (4) time to hospital discharge, (5) avoidance of inpatient care, (6) labs (all to be performed when available)—CBC, lymphocyte counts, CRP, IL-6, serum iFABP, serum lipopolysaccharide, and stool levels of viral shedding, (7) change in GI symptom scores: anorexia, diarrhea, nausea, vomiting, and abdominal pain, and (8) safety.

### Example 2: A Study to Evaluate the Safety and Efficacy of Xifaxan® (Rifaximin) Tablets in Adults with Diarrhea Associated with COVID-19

The benefits of a rifaximin therapy for diarrhea associated with COVID-19 in patients may be demonstrated with a clinical trial according to the following protocol.

The primary objective of the study is to evaluate the safety and efficacy of adding rifaximin to the current COVID-19 treatment regimen to reduce disease severity in subjects with diarrhea associated with COVID-19.

This study is a Phase 2, randomized, double-blind, placebo controlled, parallel-group interventional clinical trial to evaluate the safety and efficacy of rifaximin (Xifaxan®) in adult subjects who have confirmed or suspected COVID-19 and, as a result, are experiencing diarrhea. The study will consist of 2 arms comparing treatment with rifaximin to placebo while allowing subjects to continue receiving their current treatment regimen for COVID-19. Subjects will receive study drug for 14 days, will attend a Follow-up Visit 14 days after completion of study drug treatment (Day 28), and will be contacted by telephone for an additional follow-up 28 days later (Day 56).

The two treatment groups will be divided in Study Group A and Study Group B. Study Group A will receive Xifaxan® 550 mg tablets three times daily (TID). Study Group B will receive placebo tablets TID.

The primary endpoint will be the number of days until complete resolution of loose/watery stools (no diarrheal stools [BSFS 6 or 7] reported for at least 24 hours).

The secondary endpoints will include: (1) Change from Baseline to Day 14 in the Bristol Stool Form Scale (BSFS) type; (2) Time to improvement of one type or greater in the B SFS; and (3) Change from Baseline to Day 14 in the GI symptom severity assessment (abdominal discomfort, diarrhea, nausea, and vomiting) based on a composite score (sum of each symptom score) assessed on 10-point ordinal scale.

The exploratory endpoints will include: (1) Change from Baseline to Day 28 and Day 56 in the GI symptom severity assessment (abdominal discomfort, diarrhea, nausea, and vomiting) based on a composite score (sum of each symptom) assessed on 10-point ordinal scale; (2) Change from Baseline to Days 14, 28 and 56 in each individual GI symptom severity (abdominal discomfort, diarrhea, nausea, and vomiting); (3) Change from Baseline to Day 28 and Day 56 in the Bristol Stool Form Scale (B SFS) type; (4) Change from Baseline to Day 14, Day 28, and Day 56 in the COVID-19 symptom severity assessment (cough, shortness of breath, body ache/headache, fatigue) based on a composite score (sum of each symptom) assessed on a 10-point ordinal scale; (5) Change from Baseline to Day 14, Day 28, and Day 56 in each individual COVID-19 symptom severity (cough, shortness of breath, body ache/headache, fatigue); and (6) Rate of hospitalization.

Subjects in this study will be male or non-pregnant females ≥18 years of age that are non-hospitalized (emergency room [ER] subjects allowed) with laboratory confirmed or suspected COVID-19 (SARS-CoV-2 infection), and currently experiencing diarrhea with at least 3 loose and/or watery stools (corresponding to BSFS of 6 or 7) over previous 24 hours.

Subjects will be treated for 14 days with a Follow-up Visit 14 days after completion of treatment with study drug (Day 28) and a follow-up phone call 28 days later (Day 56).

Study Procedures. Informed consent will be obtained for potentially eligible subjects and evaluation for eligibility will begin. Subject demographics, medical history (including substance abuse, tobacco use, and a detailed history on COVID-19) and concomitant medications and procedures will be recorded. The number of watery/loose stools (BSFS 6 or 7) over the past 24 hours will be recorded. An assessment of GI symptom severity will be made and the BSFS score will be recorded based on the consistency of the subject's most recent bowel movement. The COVID-19 symptom severity assessment will be administered. An abbreviated physical examination with vital signs, height and weight will be performed. A urine pregnancy test will be performed for women of childbearing potential (WOCBP). If the subject is eligible, they will be randomized and begin treatment. Treatment will continue through Day 14 unless the subject requires hospitalization or has an AE that leads to discontinuation.

Adverse events and new or changes in concomitant medications will be recorded from the time of informed consent signing until the End of Study (EOS) Follow-up Telephone Call.

On each treatment day (Day 1 through Day 14), prior to taking their morning study drug, the subject will record the number of watery/loose stools (BSFS 6 or 7) over the past 24 hours, the severity of their GI symptoms, the BSFS type for their most recent stool, the severity of their COVID-19 symptoms, and their body temperature on a Subject Diary. Subjects will be dispensed adequate study drug to treat for 14 days and instructed to take their study drug TID for the full 14 days.

Daily recording in the Subject Diary of body temperature, the number of watery/loose stools (BSFS 6 or 7) over the past 24 hours GI symptom severity, BSFS score, and COVID-19 symptom severity will continue following completion of study drug treatment for all subjects from Day 15 through Day 27.

On Day 28 or 14 days after completion of study drug treatment, subjects will complete a follow-up visit. The number of loose/watery stools (BSFS 6 or 7) over the past 24 hours will be recorded. An assessment of GI symptom severity will be made and the BSFS score will be recorded based on the consistency of the subject's most recent bowel movement. The COVID-19 symptom severity assessment will be administered. Vital signs will be obtained, and an abbreviated physical exam will occur. A new Subject Diary will be provided.

Daily recording in the Subject Diary of body temperature, the number of watery/loose stools (BSFS 6 or 7) over the past 24 hours, GI symptom severity, BSFS score, and COVID-19 symptom severity will continue for all subjects from Day 29 through Day 55.

On Day 56, a telephone follow-up call will be made to assess the well-being of the subject and to record AEs, concomitant medications and procedures, number of watery/loose stools (BSFS 6 or 7) over the past 24 hours, GI symptom severity, BSFS, COVID-19 symptom severity, and body temperature. Data collected on the Subject Diary from Day 29 through Day 56 will be transcribed or forwarded to the clinical site.

After completion of all assessments on the Day 56 Visit, the subject will be discharged from the study.

Evaluation Criteria. Efficacy will be assessed through collection of the following: The Bristol Stool Form Scale (BSFS); Number of loose/watery stools; The GI symptom severity assessment; The COVID-19 symptom severity assessment; and Hospitalization incidence.

Safety will be assessed by collection of AEs, vital signs, abbreviated physical examinations, and urine pregnancy testing. Subjects with new onset diarrhea requiring medical attention (visit to hospital, ER, or primary care physician) during the study (through Day 56) will be tested for the presence of Clostridium difficile (C. diff) in their stool. If the test for C. diff is positive, it will be recorded as an AE of “C. diff diarrhea” and treatment with study drug will cease.

Conclusion. It is expected that this study will demonstrate, as compared to placebo, that the use of rifaximin will provide more rapid improvement in patients with diarrhea associated with COVID-19.

### Numbered Embodiments

Embodiment 1a. Rifaximin for use in a method of treating a coronavirus and/or gastrointestinal (GI) coronavirus symptoms and/or a GI symptom associated with a coronavirus in a patient in need thereof.

According to a preferred embodiment, the rifaximin is administered to the patient in a therapeutically effective amount.

Embodiment 2a. The rifaximin for use according to embodiment la, wherein the coronavirus is COVID-19.

Embodiment 3a. The rifaximin for use according to embodiment 1a or 2a, wherein the rifaximin comprises a rifaximin solvate, hydrate, polymorph, crystal, or combination thereof.

Embodiment 4a. The rifaximin for use according to any one of the preceding embodiments, wherein the rifaximin comprises rifaximin polymorph form α, rifaximin polymorph form β, rifaximin polymorph form γ, rifaximin polymorph form δ, rifaximin polymorph form ϵ, amorphous rifaximin, or a combination thereof.

Embodiment 5a. The rifaximin for use according to any one of the preceding embodiments, wherein the rifaximin is administered in an amount of about 1 mg to about 2500 mg, or about 50 mg to about 1000 mg, or about 150 mg to about 600 mg, or about 200 mg to about 550 mg, or about 200 mg, or about 550 mg.

Embodiment 6a. The rifaximin for use according to any one of the preceding embodiments, wherein the rifaximin is a 200 mg rifaximin dosage form or a 550 mg rifaximin dosage form.

Embodiment 7a. The rifaximin for use according to any one of the preceding embodiments, wherein the rifaximin is a 550 mg rifaximin dosage form.

Embodiment 8a. The rifaximin for use according to any one of the preceding embodiments, wherein rifaximin is administered QD, BID, TID, or QID.

Embodiment 9a. The rifaximin for use according to any one of the preceding embodiments, wherein rifaximin is administered TID.

Embodiment 10a. The rifaximin for use according to any one of the preceding embodiments, wherein in addition to the rifaximin an additional therapeutic agent is used.

Embodiment 11a. The rifaximin for use according to any one of the preceding embodiments, wherein the additional therapeutic agent is administered concomitantly or sequentially with the rifaximin.

Embodiment 12a. The rifaximin for use according to embodiments 10a or 11a, wherein the additional therapeutic agent comprises one or more of an antiviral and azithromycin.

Embodiment 13a. The rifaximin for use according to embodiment 12a, wherein the antiviral comprises one or more of hydroxychloroquine and chloroquine.

Embodiment 14a. The rifaximin for use according to embodiments 12a or 13a, wherein the additional therapeutic agent is administered in an amount of about 100 mg to about 1000 mg, or about 200 mg to about 900 mg, or about 300 mg to about 800 mg, or about 400 mg to about 700 mg, or about 250 mg, or about 500 mg, or about 600 mg.

Embodiment 15a. The rifaximin for use according to any one of embodiments 10a to 14a, wherein the additional therapeutic agent is administered QD, BID, TID, or QID.

Embodiment 16a. The rifaximin for use according to embodiment 15a, wherein the additional therapeutic agent is administered BID.

Embodiment 17a. The rifaximin for use according to any one of the preceding embodiments, wherein furthermore supplemental oxygen, noninvasive ventilation, invasive ventilation, additional antiviral agents, other antibiotic agents, vasopressor support, renal-replacement therapy, extracorporeal membrane oxygenation, or a combination thereof is administered to the patient.

Embodiment 18a. The rifaximin for use according to any one of the preceding embodiments, wherein the rifaximin is administered until clearance of viral infection based on a negative test for the coronavirus.

Embodiment 19a. The rifaximin for use according to any one of the preceding embodiments, wherein the rifaximin is administered until the patient no longer exhibits coronavirus symptoms.

Embodiment 20a. The rifaximin for use according to any one of the preceding embodiments, wherein the rifaximin is administered for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s).

Embodiment 21a. The rifaximin for use according to any one of the preceding embodiments, wherein the rifaximin is administered after the patient no longer exhibits coronavirus symptoms for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 additional day(s).

Embodiment 22a. A kit of parts comprising rifaximin and chloroquine for use in a method of treating coronavirus and/or gastrointestinal (GI) coronavirus symptoms and/or a GI symptom associated with coronavirus in a patient in need thereof.

According to a preferred embodiment, the rifaximin and the chloroquine are administered to the patient in a therapeutically effective amount.

Embodiment 23a. The kit of parts for use according to embodiment 22a, wherein the coronavirus is COVID-19.

Embodiment 24a. The kit of parts for use according to embodiment 22a or 23a, wherein the rifaximin is administered as a 550 mg rifaximin dosage form and the chloroquine is a 500 mg chloroquine dosage form.

Embodiment 25a. The kit of parts for use according to embodiments 22a to 24a, wherein the rifaximin is administered TID and the chloroquine is administered BID.

Embodiment 26a. The kit of parts for use according to any one of the preceding embodiments, wherein (i) the patient's heart rhythm is monitored; and/or (ii) irregularities in the patient's heart rhythm are detected.

Embodiment 27a. The kit of parts for use according to any one of the preceding embodiments, wherein the GI symptom associated with coronavirus is selected from the group consisting of nausea, anorexia, diarrhea, vomiting, abdominal pain, and a combination thereof.

Embodiment 28a. The kit of parts for use according to embodiment 27a, wherein the GI symptom associated with coronavirus is diarrhea.

Embodiment 29a. The kit of parts for use according to any one of embodiments 1a to 26a, wherein the coronavirus is COVID-19 and the GI symptom associated with coronavirus is selected from the group consisting of nausea, anorexia, diarrhea, vomiting, abdominal pain, and a combination thereof.

Embodiment 30a. The kit of parts for use according to embodiment 29, wherein the GI symptom associated with coronavirus is diarrhea.

Embodiment 31a. A kit for use in a method of treating coronavirus and/or gastrointestinal (GI) coronavirus symptoms and/or a GI symptom associated with coronavirus according to any one of embodiments 1a to 30a, wherein the kit comprises an amount of rifaximin.

Embodiment 32a. The kit for use according to embodiment 31a, wherein the coronavirus is COVID-19.

Embodiment 33a. The kit for use according to embodiment 31a or 32a, wherein the amount of rifaximin is a therapeutically effective amount

1. A method of treating a coronavirus and/or gastrointestinal (GI) coronavirus symptoms and/or a GI symptom associated with a coronavirus in a patient in need thereof, comprising administering a therapeutically effective amount of rifaximin.

2. The method of claim 1, wherein the coronavirus is COVID-19.

3. The method of claim 1, wherein the rifaximin comprises a rifaximin solvate, hydrate, polymorph, crystal, or combination thereof.

4. (canceled)

5. The method of claim 1, wherein the rifaximin is administered in an amount of about 1 mg to about 2500 mg, or about 50 mg to about 1000 mg, or about 150 mg to about 600 mg, or about 200 mg to about 550 mg, or about 200 mg, or about 550 mg.

6. (canceled)

7. (canceled)

8. The method of claim 1, wherein the rifaximin is administered QD, BID, TID, or QID.

9. (canceled)

10. The method of claim 1, further comprising administering an additional therapeutic agent.

11. The method of claim 10, wherein the additional therapeutic agent is administered concomitantly or sequentially with the rifaximin.

12. (canceled)

13. The method of claim 12, wherein the antiviral comprises one or more of hydroxychloroquine and chloroquine.

14. (canceled)

15. (canceled)

16. (canceled)

17. The method of claim 1, further comprising administering to the patient supplemental oxygen, noninvasive ventilation, invasive ventilation, additional antiviral agents, other antibiotic agents, vasopressor support, renal-replacement therapy, extracorporeal membrane oxygenation, or a combination thereof.

18. The method of claim 1, wherein the rifaximin is administered until clearance of viral infection based on a negative test for the coronavirus.

19. The method of claim 1, wherein the rifaximin is administered until the patient no longer exhibits coronavirus symptoms.

20. (canceled)

21. (canceled)

22. A method of treating coronavirus and/or gastrointestinal (GI) coronavirus symptoms and/or a GI symptom associated with coronavirus in a patient in need thereof, the method comprising administering a therapeutically effective amount of rifaximin and a therapeutically effective amount of chloroquine to the patient.

23. The method of claim 22, wherein the coronavirus is COVID-19.

24. The method of claim 22, wherein the rifaximin is administered as a 550 mg rifaximin dosage form and the chloroquine is a 500 mg chloroquine dosage form.

25. The method of claim 22, wherein the rifaximin is administered TID and the chloroquine is administered BID.

26. The method of claim 1, further comprising one or more of: (i) monitoring the patient's heart rhythm; and (ii) detecting irregularities in the patient's heart rhythm.

27. The method of claim 1, wherein the GI symptom associated with coronavirus is selected from the group consisting of nausea, anorexia, diarrhea, vomiting, abdominal pain, and a combination thereof.

28. The method of claim 27, wherein the GI symptom associated with coronavirus is diarrhea.

29. The method of claim 1, wherein the coronavirus is COVID-19 and the GI symptom associated with coronavirus is selected from the group consisting of nausea, anorexia, diarrhea, vomiting, abdominal pain, and a combination thereof.

30. (canceled)

31. A kit for treating coronavirus and/or gastrointestinal (GI) coronavirus symptoms and/or a GI symptom associated with coronavirus according to the method of claim 1, wherein the kit comprises an amount of rifaximin.

32. (canceled)

33. (canceled)
